(19) United States
(12) Patent Application Publication
(10) Pub. No.: US 2025/0041209 A1
(43) Pub. Date: Feb. 6, 2025

(54) THERMOSENSITIVE MODIFIED CHITIN SPONGE DRUG-LOADED IMPLANTED SUSTAINED-RELEASE SYSTEM, PREPARATION METHOD THEREFORE, AND USE THEREOF

(71) Applicant: Xulin JIANG, Wuhan (CN)

(72) Inventors: Xulin JIANG, Wuhan (CN); Qianqian QIAO, Wuhan (CN); Taotao LI, Wuhan (CN); Fan YANG, Wuhan (CN)

(73) Assignee: Xulin JIANG, Wuhan (CN)

(21) Appl. No.: 18/924,547

(22) Filed: Oct. 23, 2024

Related U.S. Application Data
(63) Continuation of application No. PCT/CN2023/090930, filed on Apr. 26, 2023.

(30) Foreign Application Priority Data
Apr. 27, 2022 (CN) 2022104578208

(51) Int. Cl. A61K 9/00; A61K 31/445; A61K 45/06; A61K 47/36; A61P 23/02

(52) U.S. Cl. CPC A61K 9/0024; A61K 31/445; A61K 45/06; A61K 47/36; A61P 23/02

(57) ABSTRACT

The present invention relates to a thermosensitive modified chitin sponge drug-loaded implanted sustained-release system, preparation method therefore, and use thereof. The system comprises the following components: a spongy matrix, the matrix comprising at least one thermosensitive modified chitin polymer, and at least one drug, the drug being a local anesthetic and analgesic drug and/or an anti-inflammatory and antibacterial drug, and the drug being uniformly dispersed in the matrix. All ingredients of the thermosensitive modified chitin sponge drug-loaded implanted sustained-release system have good biodegradability, biocompatibility and bioactivity, and high safety. The system provides drug sustained-release and long-lasting effect, solving the problems that the existing local anesthetic drugs have a short action time. The system may promote wound healing, being easy to store and ship, convenient to use directly, which has great potential in clinical application.

### CROSS-REFERENCE TO RELATED APPLICATIONS

The application claims priority to Chinese patent application No. 2022104578208, filed on Apr. 27, 2022, the entire contents of which are incorporated herein by reference.

### TECHNICAL FIELD

The present invention belongs to the field of pharmaceutical preparations, relates to the thermosensitive modified chitin sponge drug-loaded implanted sustained-release system for providing local analgesia, local anesthesia, and/or local anti-inflammatory as well as preparation method therefore and use thereof.

### BACKGROUND

A pain is often accompanying with substantial or potential tissue damages. An acute pain occurs after trauma or surgery and has self-limitation. The acute pain can be relieved after the tissue damage is recovered, and will be developed into chronic pain if no relieving. The chronic pain refers to a pain whose lasting time exceeds normal healing time of an acute damage or disease and which is recurred for a long term. Some long-term violent pains, such as nerve pains, cancer pains, arthritis pains and lumbagos and backaches, are not only torments that are unbearable for patients but also can cause physiological dysfunction. Thus, analgesia is one of important tasks and difficulties in clinical treatment, and is also an important subject for medical researches.

At present, postoperative analgesia mainly includes systemic analgesia and local and regional analgesia. The former mainly means oral or intravenous administration of analgesic drugs such as opioid drugs and nonsteroidal anti-inflammatory drugs (NSAIDs). Because these drugs have effects on multiple systems, especially many side effects such as constipation, nausea and vomiting, more seriously, respiratory depression and even death can be caused. In addition, long-term administration of opioid drugs can cause tolerance, drug abuse and addiction. The local analgesia mainly means local application of anaesthetic drugs (local anaesthetic drugs). The local anaesthetic drug can block the delivery of algesia signals to a central nervous system by blocking sodium and other ion channels on nerve cytomembranes so as to cause the block of electric signals of all structures at a nerve downstream, thereby achieving the effect of postoperative analgesia. The local anaesthetic drug has strong action pertinence, reduces systemic side effects and has better safety. Meanwhile, the local analgesia is simple in operation and can be completed in outpatient service, so it is cheap in cost and decreases the burden of patients. At present, the local anaesthetic drug has been applied to postoperative analgesia of surgical operations such as thoracotomy, laparotomy, cesarean section, breast cancer operation, cosmetic breast surgery and limb amputation, and plays an important role in controlling and relieving acute and chronic pains after various large-scale surgeries.

However, the existing local anaesthetic and analgesic drugs have a short action time, generally no more than a few hours, and the analgesia time required clinically is generally 24 hours, several days or more than ten days. Therefore, it is needed to increase drug dosage, repeated administration, implantation of a catheter, self-control of an analgesia pump and other technologies to prolong its analgesia effect. However, cardio cerebral side effects of these anesthetic drugs have low incidence, but they can greatly threaten the life safety of patients once occurring, the implantation of the catheter and the self-control of the analgesia pump not only needs expensive equipment and continuous guardianship which should be removed after use, but also easily causes obstruction and damage of the catheter, infection complications and other problems.

Sustained-release local anesthetic drugs can not only improve the analgesic effect of the local anesthetic drugs, but also reduce the frequency of medication and reduce adverse reactions caused by high-dose use, so they have attracted more and more attentions. At present, local anesthetic long-acting sustained-release administration systems mainly include microspheres, liposomes, implants, injectable in-situ gel, etc. Microspheres are spherical or quasi spherical tiny spherical entities. The particle size range of the microspheres is generally 1-500 microns, the small particle size can be several nanometers, and the large particle size can be up to 800 microns. The microspheres have better fluidity than irregular powder particles, but the present microspheres have broad size distribution, many drugs are deposited and crystallized on the surfaces of microspheres, so that there is an obvious phenomenon of high burst drug release, which may affect the duration of block and even produce local or systemic toxicity. In addition, the microspheres have poor in-situ performance and may be diffused along a muscle space at the injection site after injection.

There are many researches on liposomal local anesthetic drugs, in which the liposomal local anesthetic formulation Exparel®, the first sustained-release local anesthetic drug for clinical use, was approved by U.S. Food and Drug Administration (FDA) in 2011 for the first time to be applied to local infiltration anesthesia, providing an analgesic effect lasting for 72 h in human body, and was further approved to be used for brachial plexus block of intermuscular sulcus in 2018. However, one of the weaknesses of Exparel is the instability of liposomes with easy leakage of the drug, which is described as unstable “on the shelf”, needs to be stored at 2-8° C. and must be transported via a cold chain. In addition, the used carrier liposome itself may cause some granuloma inflammations. At present, only in a few countries has Exparel been approved for local anesthesia in clinical application, and this kind of liposome local anesthetic drugs have not been approved for marketing in China.

Thereby are limited researches on implants as sustained-release local anesthetic drugs. The FDA has approved on Aug. 28, 2020 that an aseptic surgical implant XARACOLL is applied to pain control after open inguinal hernia repair. XARACOLL is a unique, non-injectable drug-device combination in a form of a fully bioabsorbable collagen implant containing bupivacaine hydrochloride. XARACOLL is placed directly at the surgical site during the surgery and can release bupivacaine immediately and over time after that. Compared with placebo, XARACOLL can provide a statistically significant analgesic effect within 24 hours. However, the collagen is from animals, having a risk of viral contamination and immune allergic reactions and high cost.

Chitin and its derivatives have very good biocompatibility, biodegradability and multiple biological activities, which are suitable for drug sustained-release carriers and biomedical materials. Chitin reacts with a carboxylation reagent in a sodium hydroxide-urea aqueous system to result in carboxyl chitin with pH sensitivity and temperature sensitivity. Also thermosensitive hydroxypropyl chitin, thermosensitive hydroxybutyl chitosan and thermosensitive hydroxyamyl chitosan can be prepared from chitin in the sodium hydroxide-urea aqueous system. The thermosensitive polymers in aqueous solutions undergoing sol-gel transition due to the temperature change have been widely investigated for medical applications. In particular, thermosensitive polymer with lower critical solution temperature at lower temperature (LCST lower than body temperature) is not soluble in water at body temperature. Its aqueous solution can evenly encapsulate cells/drugs being a free flowing liquid at low temperature (e.g. 4° C.), while being transformed into hydrogel/sponge and being not soluble in water at body temperature (37° C.), so that the loaded drug molecules cannot be washed away and the drug-loaded implanted sponge system could achieve local drug sustained-release. However, there are very limited researches on preparation of long-acting local anesthetic sustained-release implanted systems by using these thermosensitive and degradable chitin derivatives. It is very important to develop new sustained-release dosage forms of long-acting local anesthetic drugs which can prolong the action time, is convenient to use and good in degradability and compatibility. Therefore, the disclosure reports a thermosensitive modified chitin sponge drug-loaded implanted sustained-release system, which is expected to be applied for long-acting local anesthesia and analgesia and/or an anti-inflammatory and antibacterial purpose, based on the thermosensitive polymers especially on the thermosensitive modified chitins.

### SUMMARY

In view of the deficiencies in the prior art in combination with the basis of previous works, the objective of the invention is to provide a thermosensitive modified chitin sponge drug-loaded implanted sustained-release system, a preparation method and use.

### Detailed Description of the Invention

The technical solution adopted by the invention is specifically as follows:

This invention relates a thermosensitive modified chitin sponge drug-loaded implanted sustained-release system, wherein the system comprises the following components: a spongy matrix, the matrix comprising at least one thermosensitive modified chitin polymer, and at least one drug, the drug being a local anesthetic and analgesic drug and/or an anti-inflammatory and antibacterial drug, and the drug being uniformly dispersed in the matrix.

Preferably, the gel transition temperature of the thermosensitive modified chitin is in the range of 1˜36° C., further preferably, the gel transition temperature of the thermosensitive modified chitin is in the range of 5˜25° C. The thermosensitive modified chitin above can be dissolved in aqueous solution, which is a flowable liquid below the gel transition temperature, will gel quickly at near body temperature, and the corresponding sponge made from the thermosensitive modified chitin is not soluble at physiological condition.

Preferably, the thermosensitive modified chitin is a combination of any one or more of thermosensitive hydroxypropyl chitin, thermosensitive carboxymethyl chitin, thermosensitive hydroxyethyl chitin, thermosensitive hydroxybutyl chitin, thermosensitive quaternized chitin or thermosensitive hydroxybutyl chitosan, the concentration of the thermosensitive modified chitin in the spongy matrix is 20-100% by mass.

Preferably, the thermosensitive modified chitin is thermosensitive hydroxypropyl chitin with molar degrees of hydroxypropyl substitution between 0.53-1.23, degree of acetylation between 0.80-0.95 and the gel transition temperature of 10-30° C.

Preferably, the thermosensitive modified chitin drug-loaded implanted sustained-release spongy matrix also contains a combination of any one or more of collagen, hyaluronic acid, polyaspartic acid, polyglutamic acid, their amount in total in the spongy matrix is 0-80% by mass. Addition of auxiliary components collagen, hyaluronic acid, polyaspartic acid or/and polyglutamic acid in the drug-loaded spongy matrix could help to retard the drug release.

Preferably, the drug-loaded sponge above contains at least one drug, the drug being a local anesthetic and analgesic drug and/or an anti-inflammatory and antibacterial drug, and the local anesthetic and analgesic drug is an amino amide anaesthetic, an amino ester anesthetics and mixtures thereof, selected from any one or more of procaine, bupivacaine, levobupivacaine, tetracaine, ropivacaine, etidocaine, articaine, lidocaine, mepivacaine, prilocaine and oxethazaine, and their salts and prodrugs.

Further preferably, the concentration of local anesthesia used is from 0.1 wt % to 5 wt %, the local anesthesia is selected from bupivacaine or ropivacaine. Further optionally, the concentration of local anesthesia used is from 0.5 wt % to 3 wt %, the local anesthesia is selected from ropivacaine hydrochloride (the drug and polymer mass fractions indicated are referred to the sponge precursor solution before freeze drying). Such drug-loaded implanted sponge system has a better long-acting sustained-release local anesthesia effect, which lasts for at least 1 day at the implanted local site or the effective blockade time of such sponge in this invention is not less than 2 times to that of ropivacaine hydrochloride solution. Further optionally, the effective blockade time of such sponge in this invention is about 3-8 times to that of ropivacaine hydrochloride solution.

Preferably, the anti-inflammatory and antibacterial drug is a non-steroidal anti-inflammatory drug, steroidal anti-inflammatory drug or an antibacterial drug, selected from any one or more of aspirin, acetaminophen, indomethacin, naproxen, nabumetone, diclofenac, ibuprofen, nimesulide, rofecoxib, celecoxib, dexamethasone, prednison acetate, cortisone.

Preferably, the spongy matrix of the thermosensitive modified chitin drug-loaded implanted sustained-release system also contains coating on the outer surface, the coating material selected from any one or more of dodecanol, tetradecanol, hexadecanol, octadecanol, dodecanoic acid, hexadecanoic acid, octadecanoic acid, the amount of coating materials in the spongy matrix is 2-20% by mass.

This invention also provides a preparation method of a thermosensitive modified chitin sponge drug-loaded implanted sustained-release system, comprising the following steps:
(1) dissolving thermosensitive modified chitin in normal saline or alkaline water below the gel transition temperature to obtain solution A;
(2) dissolving the drug or the drug hydrochloride into normal saline or acidic water to obtain solution B; and
(3) mixing the solution A with the solution B at a low temperature to prepare a mixed sponge precursor solution, and adjusting pH to 6.0-8.0 to obtain a homogenous mixed solution or even dispersion, being put in the cold storage chamber to defoam;
(4) the mixture from step (3) being added to a cubic mold for freezing and freeze drying, resulting in the thermosensitive modified chitin sponge drug-loaded implanted sustained-release system.

Preferably, the thermosensitive modified chitinin is dissolved in physiological saline solution or alkaline water at 2-30° C. in step (1); Further preferably, the thermosensitive modified chitinin is dissolved in physiological saline solution or alkaline water at 4-15° C.

Preferably, the thermosensitive modified chitin used in step (1) is thermosensitive hydroxypropyl chitin, thermosensitive carboxymethyl chitin, thermosensitive hydroxyethyl chitin, thermosensitive hydroxybutyl chitin, thermosensitive quaternized chitin or thermosensitive hydroxybutyl chitosan or their combination.

Preferably, the thermosensitive modified chitin used in step (1) is thermosensitive hydroxypropyl chitin and the concentration of the thermosensitive hydroxypropyl chitin used is from 0.5 wt % to about 6 wt %.

Further preferably, the concentration of the thermosensitive hydroxypropyl chitin used is from 1.0 wt % to about 4.0 wt %.

Preferably, dissolving thermosensitive modified chitin in alkaline water below the gel transition temperature to obtain solution A with pH around 8-14 and the concentration of the thermosensitive modified chitin from 0.5 wt % to 6 wt %. Further preferably, the alkaline water above includes one or more of aqueous NaOH solution, aqueous KOH solution, aqueous Ba(OH)₂ solution, aqueous ammonia solution and aqueous LiOH solution etc.

Preferably, the preparation step (2) specifically comprises: dissolving the drug or the drug hydrochloride into acidic water to obtain solution B with the drug concentration from 0.1 wt % to 5 wt %; Further preferably, acidic water is referred to one of the aqueous HCl solution, the aqueous H₂SO₄ solution or acetic acid solution.

Preferably, the local anesthetic and analgesic drug is an amino amide anaesthetic, an amino ester anesthetics and mixtures thereof, selected from any one or more of procaine, bupivacaine, levobupivacaine, tetracaine, ropivacaine, etidocaine, articaine, lidocaine, mepivacaine, prilocaine and oxethazaine, and their salts and prodrugs.

Preferably, the preparation step (3) specifically comprises: mixing the solution A with the solution B at a low temperature to prepare a mixed sponge precursor solution, and adjusting pH to 7.0-7.5 to obtain a homogenous mixed solution or even dispersion, being put in the cold storage chamber to defoam. The prepared neutral sponge with pH adjustment showed much better and obvious long-lasting sustained-release effect as indicated in Example 3 of this invention.

Preferably, the preparation step (4) may specifically comprises coating step wherein the coating on the sponge outer surface is sprayed by a liquid layer, which can be dried or solidified to form a solid thin layer on the spongy matrix.

Further preferably, wherein the coating material used is a homogeneous liquid after heating or dissolving in volatile solvent such as ethanol, which can form a solid thin layer on the spongy matrix by cooling to solidify from liquid to solid state or by evaporation to remove solvent. The coated sponge has fewer drug burst release and is more long-lasting and slowly sustained release than the uncoated sponge as indicated in Example 2 of this invention.

This invention also provides use of the thermosensitive modified chitin sponge drug-loaded implanted sustained-release system for a drug for long-acting local anesthesia, analgesia, anti-inflammation and itching relieving.

Preferably, the invention provides use of the drug-loaded implanted sponge for a drug for block of peripheral nerves or control of postoperative wound pain at least 1 day after operation.

Further preferably, use of such drug-loaded implanted sponge system for control of postoperative wound pain, which lasts for at least 1 day at the implanted local site or the effective blockade time of such sponge in this invention, is not less than 2 times to that of ropivacaine hydrochloride solution. Further optionally, the effective blockade time of such sponge in this invention is about 3-8 times to that of ropivacaine hydrochloride solution.

Preferably, the drug-loaded implanted sponge in this invention can be applied for pain control of long-acting local anesthetic and analgesic drug or anti-inflammatory and antibacterial drug sustained-release at least 1 day after local surgical operations, including but not limited to, laparotomy, orthopaedic surgery, gynecologic surgery, thoracotomy, or abdominal surgery. It will be implanted subcutaneously before suturing after operation, such as open inguinal hernia repair, abdominal hernia repair, abdominal plastic surgery, for pain control of long-acting local anesthetic and analgesic or anti-inflammatory and antibacterial purpose.

Below is the principle of this invention:

This invention is based on that thermosensitive polymers such as thermosensitive modified chitins are soluble in water at low temperature (lower than the gel transition temperature or lower critical solution temperature (LCST) of thermosensitive modified chitin, polymer concentration in aqueous solution required to have LCST is lower here in the range of 0.5-6 wt %), the local anesthetic and analgesic drug being evenly encapsulated and being freeze dried to obtain the drug loaded sponge, which is not soluble in water at body temperature (37° C., the gel transition temperature or LCST of thermosensitive modified chitin lower than body temperature), so that the loaded drug molecules can only come out through slow-diffusion not being washed away and the drug-loaded implanted sponge system could achieve the purpose of the local drug sustained-release.

Compared with the prior art, this invention has the beneficial effects:
1. The thermosensitive modified chitin material used in this invention has good biodegradability, biocompatibility and bioactivity and high safety, which can load the drug and release the drug slowly, thereby prolonging the analgesia time of the local anesthetic drug (the effective blockade time of the thermosensitive modified chitin sponge drug-loaded implanted sustained-release system in this invention is about 3-8 times to that of direct use of the local anesthetic drug in solution such as ropivacaine hydrochloride solution), solving the problems that the existing local anesthetic drugs have a short action time. The drug loaded spongy system may promote the wound healing, being easy to store and ship, convenient to use directly, which has great potential in clinical application.
2. The preparation process of the thermosensitive modified chitin sponge drug-loaded implanted sustained-release system in this invention is simple, it does not use toxic organic solvents and chemical crosslinking agents and has no problems of chemical residual, the cost is low, and the environment is free from pollution, which is suitable for large scale production.

### BRIEF DESCRIPTION OF THE DRAWINGS

FIG. 1 is the bupivacaine (B) cumulative release profiles in example 1 of this invention in a PBS buffer solution, from the sponges of 1 (0.8% B-collagen (0.6 wt %)), 2 (0.8% B-HPCH (2.0 wt %)), 3 (0.8% B-HPCH (3.6 wt %)), 4 (0.8% B-collagen/HPCH (0.6/3.0 wt %)) with 0.8% drug loading content, 5 (1.2% B-HPCH (3.6 wt %)) and 6 (1.2% B-collagen/HPCH (0.6/3.0 wt %)) with 1.2% drug loading content, and 7 (2.4% B-HPCH (3.6 wt %)) and 8 (2.4% B-collagen/HPCH (0.6/3.0 wt %)) with 2.4% drug loading content, respectively. Data are presented as mean±SD, n=3.

FIG. 2 is the ropivacaine (R) cumulative release profiles in example 2 of this invention in a PBS buffer solution, from the sponges of 1 (0.8% R-collagen/HPCH (0.6/3.0 wt %)) and 2 (0.8% R-collagen/HPCH (0.6/3.0 wt %)-TDC) with 0.8% drug loading content. Data are presented as mean±SD, n=3.

FIG. 3 is the ropivacaine (R) cumulative release profiles in example 4 of this invention in a PBS buffer solution, from the sponges of 1 (0.8% R-collagen (0.6 wt %)), 2 (0.8% R-HPCH (3.6 wt %)) and 3 (0.8% R-collagen/HPCH (0.6/3.0 wt %)) with 0.8% drug loading content. Data are presented as mean±SD, n=3.

FIG. 4 is the in vivo nociceptive blockade score (a) and effective duration time of blockade (b) of 1 (0.8%-BPV solution) and sponges 2 (BPV-loaded 0.8%-HPCH (3.6 wt %)), 3 (BPV-loaded 0.8%-collagen/HPCH (0.6/3.0 wt %)), 4 (BPV-loaded 1.2%-HPCH (3.6 wt %)) and 5 (BPV-loaded 2.4%-HPCH (3.6 wt %)) in example 5 of this invention. Data are presented as mean±SD, n=6. **p<0.01, ***p<0.001.

### DETAILED DESCRIPTION OF EMBODIMENTS

For more easily understanding the present invention, the specific embodiments of the present invention will be illustrated in detail below in combination with embodiments and drawings, but these embodiments do not limit the protective scope of the disclosure in any manners.

Preparation of the thermosensitive modified chitins used in the present invention:

Thermosensitive chitin derivative hydroxypropyl chitin with low deacetylation degrees was prepared homogeneously in a sodium hydroxide-urea aqueous solution system by using a homogeneous phase method. A specific preparation method was as follows: 2 g of purified chitin was weighed and dispersed into 100 g of pre-frozen aqueous solution containing 11 wt % sodium hydroxide and 4 wt % urea under the condition of stirring to be frozen for 24 h at −30° C., then the above chitin dispersion was taken out and unfrozen at room temperature by mechanically stirring to obtain a dissolved chitin aqueous solution. 11.4 g of 1,2-propylene oxide was added into the obtained chitin solution (100 g, 2 wt %), this system was stirred at 2° C. so that reactants were evenly mixed, the reactants reacted for 24 h after the temperature was raised to 5° C. and then reacted for 6 h after the temperature was raised to 15° C. Finally, the system was cooled to 2° C., the pH value was adjusted to 7 using 3M hydrochloric acid, the system was dialyzed with deionized water and freeze-dried to obtain white sponge-like hydroxypropyl chitin (HPCH), with a yield of 87%.

Based on the ¹H NMR spectrum of HPCH, the degree of acetylation (DA) and the molar degree of substitution of hydroxypropyl (MS) of the product were 0.89 and 0.84, respectively. The viscosity-average molecular weight of HPCH was about 410 kDa obtained from a Ubbelohde viscometer. This homogeneously synthesized HPCH exhibited reversible thermosensitive sol-gel transition examined by rheological study, wherein the temperature of gelation (Tgel), indicating the sol-gel phase transition for the 2 wt % HPCH solution was 18° C. Different molar degrees of hydroxypropyl substitution (MS between 0.53-1.23) of thermosensitive HPCH samples with low degree of deacetylation (DA between 0.80-0.95) were obtained when different amount of propylene oxide (different molar feed ratio of propylene oxide to saccharide unit) was used under different reaction condition and their molecular weights ranged from 5 kDa to 1000 kDa.

Thermosensitive carboxymethyl chitin with low deacetylation degrees, were prepared homogeneously in a sodium hydroxide-urea aqueous solution system by using a homogenous phase method. 2 g of purified chitin was weighed and dispersed into 100 g of pre-frozen aqueous solution containing 11 wt % sodium hydroxide and 4 wt % urea under the condition of stirring to be frozen for 6 h at −20° C., then the above chitin dispersion was taken out and unfrozen at room temperature by mechanically stirring and then frozen and unfrozen twice repeatedly to obtain a dissolved chitin aqueous solution. 5.7 g of sodium chloroacetate was slowly added into the obtained chitin solution (100 g, 2 wt %), mechanical stirring was kept so that reactants can evenly react for 24 h at 5° C., then this system was cooled to 2° C., the pH value was adjusted to 7 using 3M hydrochloric acid, the obtained neutral solution was dropwise added into acetone so that a product was precipitated out. The obtained precipitate was washed with 80% ethyl alcohol to remove salts and urea therein. After washing, the precipitate was dried at 60° C. to obtain white powdered carboxymethyl chitin with a yield of 87%. Base on the ¹H NMR spectrum, the degree of acetylation (DA) and the degree of substitution of carboxymethyl (DS) of the obtained product CMCH were 0.82 and 0.13, respectively. The homogeneously synthesized CMCH solution is thermosensitive and pH-sensitive, can be dissolved under the conditions of a low temperature and alkaline water, and generates gelation transition under the conditions of a raised temperature and reduced alkalinity. By changing the dosage of sodium chloroacetate and controlling the molar ratio of sodium chloroacetate to sugar unit in chitin structure, a series of CMCH products with different substitution degrees (0.07-0.23) were obtained. A series of carboxymethyl chitins with different substitution degrees and low deacetylation degrees (0.72-0.92) can be prepared by changing different molar feed ratios of sodium chloroacetate to saccharide unit and reaction conditions, and their molecular weights range from 5 kDa to 1000 kDa.

Similarly, thermosensitive hydroxyethyl chitin and thermosensitive hydroxybutyl chitin with low deacetylation degrees were prepared in the sodium hydroxide-urea aqueous solution system by using the homogeneous phase method. These chitin derivatives have an acetylation degree of 0.7-0.92 and a molecular weight of 5 kDa-1000 kDa.

Example 1 Thermosensitive hydroxypropyl chitin sponge drug-loaded implanted sustained-release system and preparation method:

Local anesthetic drug bupivacaine hydrochloride (40 mg) was dissolved in 4.78 g deionized water firstly at room temperature and the solution was cooled down at 10° C. Then 180 mg of the thermosensitive hydroxypropyl chitin (HPCH with MS: 0.76, DA: 0.91, Tgel: 20° C. for 2 wt %) white sponge prepared above was added to the bupivacaine (BPV) solution. The mixture was stirred for dissolution and put in the refrigerator overnight at 4° C. to defoam. Next, the homogeneous mixture viscous liquid flowable solution (0.5 g) was added to a cubic mold of 1 cm×1 cm×1 cm for freezing and freeze drying (temperature from −22 to −60° C.), resulting in BPV-loaded 0.8%-HPCH (3.6 wt %) drug-loaded implanted sustained-release sponges (the percentage before the hyphen indicated the mass fraction of BPV, polymer concentration of 3.6 wt % in the mixture containing water), named as 0.8% B-HPCH (3.6 wt %) sponge for short.

Similarly, 60 mg, 80 mg, 120 mg or 160 mg of local anesthetic drug bupivacaine hydrochloride were used to prepare 1.2% BHCL-HPCH (3.6 wt %), 1.6% BHCL-HPCH (3.6 wt %), 2.4% BHCL-HPCH (3.6 wt %), 3.2% BHCL-HPCH (3.6 wt %) drug-loaded implanted sustained-release sponges, which were 1.2% B-HPCH (3.6 wt %), 1.6% B-HPCH (3.6 wt %), 2.4% B-HPCH (3.6 wt %), 3.2% B-HPCH (3.6 wt %) for short. Similarly, 100 mg of the thermosensitive HPCH white sponge in place of 180 mg HPCH was added to the bupivacaine solution containing bupivacaine hydrochloride 40 mg to prepare 0.8% BHCL-HPCH (2.0 wt %) drug-loaded implanted sustained-release sponge, named as 0.8% B-HPCH (2.0 wt %) for short.

As control group, BPV-loaded collagen (0.6 wt %) sponge was prepared similarly as BPV-loaded HPCH sponges except that collagen solution was added into the bupivacaine hydrochloride solution, named as 0.8% B-collagen (0.6 wt %) for short. The mass fractions indicated were referred to the sponge precursor solution before freeze drying, the percentage before the hyphen indicated the mass fraction of bupivacaine hydrochloride (B). Similarly, using collagen solution and HPCH in place of only HPCH, BPV-loaded collagen-HPCH sponge containing 0.6 wt % collagen and 3.0 wt % HPCH was prepared, named as 0.8% B-collagen/HPCH (0.6/3.0 wt %) for short. Using sodium hyaluronate solution in place of collagen, BPV-loaded HA/HPCH sponge containing 0.6 wt % sodium hyaluronate and 3.0 wt % HPCH was prepared, named as 0.8% B-HA/HPCH (0.6/3.0 wt %) sponge. Similarly, adding various drug amount, various drug-loaded implanted sustained-release sponges 1.2% B-collagen/HPCH (0.6/3.0 wt %) and 2.4% B-collagen/HPCH (0.6/3.0 wt %) were prepared. It is noted that the drug and polymer mass fractions indicated were referred to the sponge precursor solution before freeze drying, unless otherwise specified. The different formulations of bupivacaine-loaded sponges were shown in Table 1 of this invention.

TABLE 1
The different formulations of bupivacaine-loaded sponges.

| | bupivacaine | | |
|---|---|---|---|
| Name/mass fractions | hydrochloride | Collagen | HPCH |
| before freeze drying | (wt %) | (wt %) | (wt %) |
| 0.8% B-collagen (0.6 wt %) | 0.8 | 0.6 | 0 |
| 0.8% B-HPCH(2.0 wt %) | 0.8 | 0 | 2.0 |
| 0.8% B-collagen/HPCH | 0.8 | 0.6 | 3.0 |
| (0.6/3.0 wt %) | | | |
| 0.8% B-HPCH (3.6 wt %) | 0.8 | 0 | 3.6 |
| 1.2% B-HPCH (3.6 wt %) | 1.2 | 0 | 3.6 |
| 1.6% B-HPCH (3.6 wt %) | 1.6 | 0 | 3.6 |
| 2.4% B-HPCH (3.6 wt %) | 2.4 | 0 | 3.6 |
| 3.2% B-HPCH (3.6 wt %) | 3.2 | 0 | 3.6 |

The BPV-loaded HPCH, collagen or collagen/HPCH cuboid sponge (volume of 0.5 mL with surface of 1 cm×1 cm) prepared above was put into a dialysis bag (molecular weight cut off: 8000-14000 Da) and immersed in 15 mL PBS (0.13M phosphate buffered saline solution, pH 7.4) in a shaking water bath (37° C., 75 rpm). At predetermined intervals, 2.0 mL of the released medium was taken out and replaced by 2.0 mL fresh buffer solution. The concentration of BPV in the released medium was calculated by UV absorbance at 263 nm using a UV-vis spectrophotometer (LAMBDA 35, PerkinElmer). The PBS solution was used as blank control, the drug concentration of bupivacaine was calculated according to a standard curve of bupivacaine hydrochloride, and the cumulative release curve was plotted. Three samples in each group were set for repetition. As control group, BPV-loaded collagen (0.6 wt %) sponge 0.8% B-collagen (0.6 wt %) containing 0.8% bupivacaine hydrochloride and 0.6 wt % collagen was used as the generic formulation of Xaracoll®.

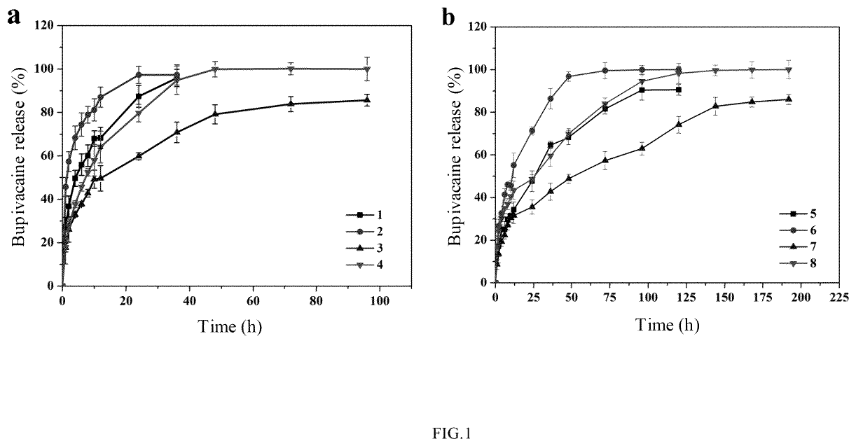

FIG. 1a is the bupivacaine (B) cumulative percentage release curves in a PBS buffer solution, from the sponges of 1 (0.8% B-collagen (0.6 wt %), as control), 2 (0.8% B-HPCH (2.0 wt %)), 3 (0.8% B-HPCH (3.6 wt %)), 4 (0.8% B-collagen/HPCH (0.6/3.0 wt %)) with 0.8% drug loading content, respectively. Data are presented as mean±SD, n=3. It can be seen clearly from this FIG. 1a that the BPV release was slower with increasing the thermosensitive HPCH concentration and BPV was released more slowly with less initial burst release from the BPV-loaded 0.8%-HPCH (3.6 wt %) sponge than that from the BPV-loaded 0.8%-collagen/HPCH (0.6/3.0 wt %) sponge with the same total polymer concentration.

FIG. 1b is the bupivacaine (B) cumulative percentage release curves in a PBS buffer solution, from the sponges of 5 (1.2% B-HPCH (3.6 wt %)) and 6 (1.2% B-collagen/HPCH (0.6/3.0 wt %)) with 1.2% drug loading content, and 7 (2.4% B-HPCH (3.6 wt %)) and 8 (2.4% B-collagen/HPCH (0.6/3.0 wt %)) with 2.4% drug loading content, respectively. Data are presented as mean±SD, n=3. It can be seen from this figure (FIG. 1b) that the release time of BPV increased from 120 h to 192 h with increasing the drug loading content from 1.2% to 2.4%. BPV-loaded 2.4%-HPCH (3.6 wt %) sponge had the most obvious sustained release effect, and its release time (192 h) was about 8 times longer than that (24 h) of control group BPV-loaded 0.8%-collagen (0.6 wt %) sponge. Thus, we could draw a conclusion that the drug-loaded implanted thermosensitive HPCH sponge system showing obvious long sustained release time has great potential in clinical analgesia application due to its high drug loading content.

Example 2 The effect of the surface coating on the thermosensitive hydroxypropyl chitin and collagen sponge ropivacaine-loaded implanted sustained-release system:

Local anesthetic drug ropivacaine hydrochloride (80 mg) was added into 7.5 g of 0.8 wt % collagen solution (pH 4) with stirring and addition of 2.12 g deionized water for dissolution firstly at room temperature and the solution was cooled down at 10° C. Then 300 mg of the thermosensitive hydroxypropyl chitin (HPCH with MS: 0.84, DA: 0.89, Tgel: 20° C. for 2 wt %) white sponge prepared above was added to the ropivacaine-collagen solution. The mixture was stirred for dissolution and put in the refrigerator overnight at 4° C. to defoam. Next, the homogeneous mixture viscous liquid flowable solution (0.9 g) was added to a tissue culture plate 24 (diameter 15.6 mm) for freezing and freeze drying (temperature from −22 to −60° C.), resulting in ropivacaine-loaded 0.8%-collagen/HPCH (0.6/3.0 wt %) drug-loaded implanted sustained-release sponge (the percentage before the hyphen indicated the mass fraction of ropivacaine hydrochloride, polymer concentration of collagen 0.6 wt % and HPCH 3.0 wt % in the mixture indicated were referred to the sponge precursor solution before freeze drying), named as 0.8% R-collagen/HPCH (0.6/3.0 wt %) sponge for short.

Similarly, as control group using collagen solution only without HPCH, local anesthetic drug ropivacaine hydrochloride (80 mg) was added into 7.5 g of 0.8 wt % collagen solution (pH 4) with stirring and addition of 1 g and 1.42 g deionized water for dissolution at room temperature (up to total weight of 10 g). The mixture was stirred evenly and the solution (0.9 g) was added to a tissue culture plate 24 (diameter 15.6 mm) for freezing and freeze drying, resulting in ropivacaine-loaded 0.8%-collagen (0.6 wt %) drug-loaded implanted sustained-release sponge, named as 0.8% R-collagen (0.6 wt %) sponge for short.

1-Tetradecanol solution (10 mg/mL, 2 mL) in ethanol prepared was sprayed evenly on the outer surface of the drug-loaded sponge (diameter 15.6 mm) made above by ultrasonic spray deposition method. After drying naturally to evaporate ethanol, the weight of each drug-loaded sponge increased around 4 mg, the ropivacaine-loaded 0.8%-collagen (0.6 wt %) drug-loaded implanted sustained-release sponge coated with tetradecanol and the ropivacaine-loaded 0.8%-collagen/HPCH (0.6/3.0 wt %) drug-loaded implanted sustained-release sponge coated with tetradecanol was named as 0.8% R-collagen (0.6 wt %)-TDC and 0.8% R-collagen/HPCH (0.6/3.0 wt %)-TDC, respectively, of which the in vitro drug release test was carried out.

The ropivacaine-loaded sponge (diameter 15.6 mm) prepared above was put into a dialysis bag (molecular weight cut off: 8000-14000 Da) and immersed in 15 mL PBS (0.13M phosphate buffered saline solution, pH 7.4) in a shaking water bath (37° C., 75 rpm). At predetermined intervals, 2.0 mL of the released medium was taken out and replaced by 2.0 mL fresh buffer solution. The drug concentration in the released medium was calculated by UV absorbance at 262 nm using a UV-vis spectrophotometer. The PBS solution was used as blank control, the drug concentration of ropivacaine was calculated according to a standard curve of ropivacaine hydrochloride, and the cumulative release curve was plotted. Three samples in each group were set for repetition.

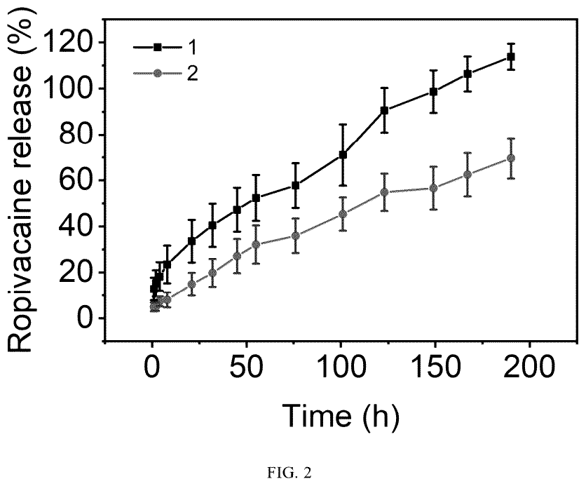

FIG. 2 shows the representative ropivacaine (R) cumulative release profiles in a PBS buffer solution, from the sponges of 1 (0.8% R-collagen/HPCH (0.6/3.0 wt %)) and 2 (0.8% R-collagen/HPCH (0.6/3.0 wt %)-TDC) with 0.8% drug loading content. Data are presented as mean±SD, n=3. It can be seen from this figure that the ropivacaine-loaded 0.8% R-collagen/HPCH (0.6/3.0 wt %) sponge shows good drug sustained-release effect, but the ropivacaine-loaded 0.8% R-collagen/HPCH (0.6/3.0 wt %)-TDC sponge coated with tetradecanol has fewer drug burst release and is more long-lasting and slowly sustained release than the uncoated sponge.

Similar test results indicated that the sustained-release effect of the ropivacaine-loaded 0.8% R-collagen (0.6 wt %)-TDC sponge coated with tetradecanol was better than that of the uncoated 0.8% R-collagen (0.6 wt %) sponge.

Similarly, using dodecanol, hexadecanol, octadecanol, tetradecanol, lauric acid, palmitic acid, stearic acid and a combination of any one or more of them such as a combination of lauric acid and stearic acid at weight ratio of 4:1, in place of 1-tetradecanol for coating, the ropivacaine-loaded thermosensitive HPCH and/or collagen implanted sustained-release sponge coated can be prepared.

Example 3 The effect of the pH value of the drug-loaded sponge dispersion on the thermosensitive hydroxypropyl chitin and collagen sponge ropivacaine-loaded implanted sustained-release system:

pH Adjustment and neutralization: local anesthetic drug ropivacaine hydrochloride (80 mg) was added into 7.5 g of 0.8 wt % collagen solution (pH 4) with stirring and addition of 1.0 g deionized water for dissolution and then 0.51 mL 1.0 M NaOH solution (0.52 g) was added slowly to adjust pH to 7.0 (neutral) with addition to 0.9 g deionized water (up to total weight of 10 g). The mixture was stirred evenly to obtain opalescent dispersion flowable liquid, from which 0.9 g was added to a tissue culture plate 24 (diameter 15.6 mm) for freezing and freeze drying, resulting in ropivacaine-loaded 0.8%-collagen (0.6 wt %) drug-loaded implanted sustained-release neutral sponge, named as 0.8% R-collagen (0.6 wt %)-pH7.

No pH adjustment for the drug-loaded collagen sponge dispersion: As control group, local anesthetic drug ropivacaine hydrochloride (80 mg) was added into 7.5 g of 0.8 wt % collagen solution (pH 4) with stirring and addition of 1 g and 1.42 g deionized water for dissolution at room temperature (up to total weight of 10 g). The mixture was stirred evenly and the solution (0.9 g) was added to a tissue culture plate 24 (diameter 15.6 mm) for freezing and freeze drying, resulting in ropivacaine-loaded 0.8%-collagen (0.6 wt %) drug-loaded implanted sustained-release sponge, named as 0.8% R-collagen (0.6 wt %) sponge for short.

The ropivacaine-loaded sponge (diameter 15.6 mm) prepared above was put into a dialysis bag (molecular weight cut off: 8000-14000 Da) and immersed in 15 mL PBS (pH 7.4) in a shaking water bath (37° C., 75 rpm). At predetermined intervals, 2.0 mL of the released medium was taken out and replaced by 2.0 mL fresh buffer solution. The drug concentration in the released medium was calculated by UV absorbance at 262 nm using a UV-vis spectrophotometer. The PBS solution was used as blank control, the drug concentration of ropivacaine was calculated according to a standard curve of ropivacaine hydrochloride, and the cumulative release curve was plotted. Three samples in each group were set for repetition. In vitro release of the ropivacaine-loaded 0.8% R-collagen (0.6 wt %) sponge with no pH adjustment showed obvious initial burst release phenomenon, the drug release was 59±4 wt % at 1 h, 80±2 wt % at 8 h, and almost 100% after 22 h. In contrast, the ropivacaine-loaded 0.8% R-collagen (0.6 wt %)-pH7 neutral sponge with pH adjustment showed obvious sustained-release effect, the drug release was 24±4 wt % at 1 h, 38±5 wt % at 8 h, about 60% after 22 h and complete release after 96 h. This indicated that the acidity in the ropivacaine-loaded collagen containing sponge could lead to the initial local anesthetic drug burst release phenomenon, but the ropivacaine-loaded collagen containing neutral sponge with pH adjustment would show good drug sustained-release effect.

pH Adjustment and neutralization: Local anesthetic drug ropivacaine hydrochloride (80 mg) was added into 7.5 g of 0.8 wt % collagen solution (pH 4) with stirring and addition of 1.0 g deionized water for dissolution firstly at room temperature and the solution was cooled down at 10° C. Then 300 mg of the thermosensitive hydroxypropyl chitin (HPCH with MS: 0.84, DA: 0.89, Tgel: 20° C. for 2 wt %) white sponge prepared above was added to the ropivacaine-collagen solution. The mixture was stirred for dissolution and put in the refrigerator overnight at 4° C. to defoam. Then 0.51 mL 1.0 M NaOH solution (0.52 g) was added slowly to adjust pH to 7.0 (neutral) with addition to 0.6 g deionized water (up to total weight of 10 g). The mixture was stirred evenly to obtain opalescent dispersion flowable liquid, from which 0.6 g at around 10° C. was added to a cubic mold of 1 cm×1 cm×1 cm for freezing and freeze drying, resulting in ropivacaine-loaded 0.8%-collagen/HPCH (0.6/3.0 wt %) drug-loaded implanted sustained-release neutral sponge, named as 0.8% R-collagen/HPCH (0.6/3.0 wt %)-pH7.

In vitro drug release comparison between the ropivacaine-loaded 0.8% R-collagen/HPCH (0.6/3.0 wt %) sponge with no pH adjustment and ropivacaine-loaded 0.8%-collagen/HPCH (0.6/3.0 wt %)-pH7 neutral sponge with pH adjustment showed that the cumulative release percentage of the former with good sustained-release effect, obvious initial burst release phenomenon, was 13±5 wt % at 1 h, 23±8 wt % at 8 h, 41±9 wt % at 32 h, 91±10 wt % at 123 h, and almost 100% after 150 h. In contrast, the latter ropivacaine-loaded 0.8%-collagen/HPCH (0.6/3.0 wt %)-pH7 neutral sponge with pH adjustment showed more obvious sustained-release effect, the cumulative release percentage was 1±1 wt % at 1 h, 4±wt % at 8 h, 20±3 wt % at 24 h, 23±3 wt % at 34 h, 75±4 wt % at 120 h, 95±3 wt % at 144 h and complete release after 160 h. This indicated that the ropivacaine-loaded collagen and HPCH containing sponge showed better sustained-release effect than that of collagen containing only sponge without HPCH and the ropivacaine-loaded collagen and HPCH containing neutral sponge with pH adjustment showed much better and obvious long-lasting sustained-release effect.

Later in this invention, the prepared drug-loaded collagen containing sponge is referred to the neutral sponge with pH adjustment, unless otherwise specified.

### Example 4 The Thermosensitive Hydroxypropyl Chitin Sponge Ropivacaine-Loaded Implanted Sustained-Release System

Using the thermosensitive hydroxypropyl chitin (HPCH with MS 0.84, DA 0.89, Tgel: 18° C. for 2 wt %) prepared above, similar to the preparation process of the ropivacaine-loaded 0.8%-collagen/HPCH (0.6/3.0 wt %)-pH7 neutral sponge with pH adjustment in Example 3, 120 mg, 160 mg, 240 mg or 320 mg of local anesthetic drug ropivacaine hydrochloride were used in place of ropivacaine hydrochloride (80 mg) to prepare 1.2% R_{HCL}-collagen/HPCH (0.6/3.0 wt %)-pH7, 1.6% R_{HCL}-collagen/HPCH (0.6/3.0 wt %)-pH7, 2.4% R_{HCL}-collagen/HPCH (0.6/3.0 wt %)-pH7, 3.2% R_{HCL}-collagen/HPCH (0.6/3.0 wt %)-pH7 ropivacaine-loaded collagen/HPCH neutral sponges with pH adjustment, named as 1.2% R-collagen/HPCH, 1.6% R-collagen/HPCH, 2.4% R-collagen/HPCH, 3.2% R-collagen/HPCH for short, respectively.

Similar to the preparation process of the bupivacaine-loaded 0.8% B-HPCH (3.6 wt %) sponge in Example 1, only using thermosensitive hydroxypropyl chitin HPCH, not using collagen solution, using ropivacaine hydrochloride in place of bupivacaine hydrochloride, a series of ropivacaine-loaded 0.8% R-HPCH (3.6 wt %), 1.2% R-HPCH (3.6 wt %), 1.6% R-HPCH (3.6 wt %), 2.4% R-HPCH (3.6 wt %) and 3.2% R-HPCH (3.6 wt %) sponges were prepared. Similar to the preparation process of the bupivacaine-loaded 0.8% B-HA/HPCH (0.6/3.0 wt %) sponge in Example 1, Using sodium hyaluronate solution in place of collagen, ropivacaine-loaded HA/HPCH sponge containing 0.6 wt % sodium hyaluronate and 3.0 wt % HPCH was prepared, named as 0.8% R-HA/HPCH (0.6/3.0 wt %) sponge.

The ropivacaine-loaded cuboid sponge (volume of 0.6 mL about 0.6 g with surface of 1 cm×1 cm) prepared above was put into a dialysis bag (molecular weight cut off: 8000-14000 Da) and immersed in 15 mL PBS (pH 7.4) in a shaking water bath (37° C., 75 rpm). At predetermined intervals, 5.0 mL of the released medium was taken out and replaced by 5.0 mL fresh buffer solution. The concentration of ropivacaine in the released medium was calculated by UV absorbance at 262 nm using a UV-vis spectrophotometer. The PBS solution was used as blank control, the drug concentration of ropivacaine was calculated according to a standard curve of ropivacaine hydrochloride, and the cumulative release curve was plotted. Three samples in each group were set for repetition.

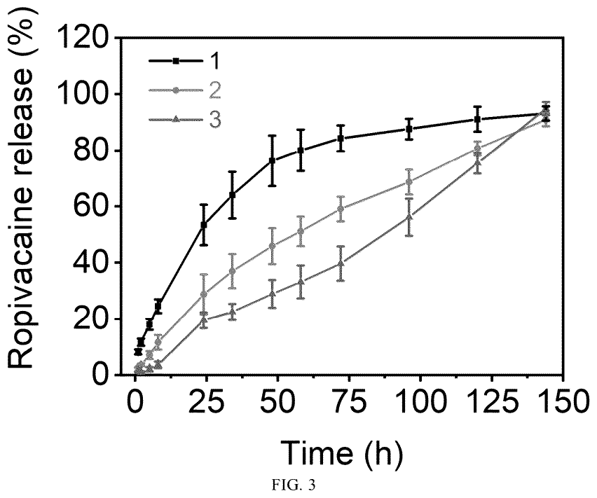

FIG. 3 is the ropivacaine (R) cumulative release profiles in a PBS buffer solution from the sponges of 1 (0.8% R-collagen (0.6 wt %) prepared in Example 3), 2 (0.8% R-HPCH (3.6 wt %)) and 3 (0.8% R-collagen/HPCH (0.6/3.0 wt %) prepared in Example 3) with 0.8% ropivacaine hydrochloride drug loading content. Data are presented as mean±SD, n=3. As seen from this figure, The control group 0.8% R-collagen (0.6 wt %) showed some sustained-release effect, but the 0.8% R-HPCH (3.6 wt %) sponge showed obviously better sustained-release effect, more slowly with less initial burst release than the control group due to use of thermosensitive HPCH. It also can be seen from FIG. 3 that ropivacaine was released much more slowly with much less initial burst release from the ropivacaine-loaded 0.8% R-collagen/HPCH (0.6/3.0 wt %) collagen/HPCH composite sponge than that from the ropivacaine-loaded 0.8% R-HPCH (3.6 wt %) HPCH-containing only sponge with the same total polymer concentration. It should be noted that the prepared drug-loaded collagen containing sponge is referred to the neutral sponge with pH adjustment here (for both groups 1 and 3 here, but not for group 2). This indicated that the ropivacaine-loaded collagen and HPCH containing neutral sponge with pH adjustment showed much better and obvious long-lasting sustained-release effect again. Similarly, Using sodium hyaluronate solution in place of collagen solution, the local anesthetic drug release from the drug loaded implanted sponge could be prolonged, but the drug loaded implanted sponge prepared containing excipient sodium hyaluronate only showed limited sustained-release effect with obvious initial burst release. When the ropivacaine hydrochloride drug loading content was increased from 0.8% to 5.0%, the drug release time of the thermosensitive hydroxypropyl chitin sponge ropivacaine-loaded implanted sustained-release system can also be prolonged clearly.

### Comparative Example 1 Other Thermosensitive Polymer Sponge Drug-Loaded Implanted Sustained-Release System

Using commercial available thermosensitive poly(ethylene glycol)-block-poly(propylene glycol)-block-poly(ethylene glycol) (PEO-PPO-PEO, Pluronic® F-127, Sigma, Product No.: P2443-250G) or thermosensitive PLGA-PEG-PLGA (Guangzhou Tanshui technology Co., Ltd, Temperature of gelation (Tgel): 15±2° C., Product No.: 81000030, Lot NO.: TS092917), the sustained drug release time in vitro for the thermosensitive polymer (PEO-PPO-PEO or PLGA-PEG-PLGA concentration of 20 wt %) sponge bupivacaine-loaded implanted sustained-release system showing some sustained-release effect was less than 16 h. It is noted that for these thermosensitive polymers such as PEO-PPO-PEO or PLGA-PEG-PLGA, the polymer concentration above 10 wt %, normally in the range of 15-30 wt %, is required to have suitable LCST near the body temperature. For example, because the softening point of PLGA-PEG-PLGA is quite low, the 0.8% ropivacaine hydrochloride drug loaded PLGA-PEG-PLGA sponge after freeze dry which could not maintain the solid shape at 37° C. showed very limited sustained-release effect. The in vitro cumulative release percentage of the 0.8% bupivacaine hydrochloride drug loaded F127 sponge with 20% polymer concentration was 38 wt % at 1 h, 47 wt % at 2 h, 87 wt % at 6 h, and almost 100% after 8 h, showing very limited sustained-release effect, which was a little inferior to that of 0.8% B-collagen (0.6 wt %) sponge (74 wt % at 6 h, 79 wt % at 8 h, and almost 100% after 24 h) and far inferior to that of 0.8% B-HPCH (3.6 wt %) sponge.

### Example 5 Long-Term Local Anesthetic Efficacy and Safety In Vivo Evaluation of the Drug-Loaded Implanted Thermosensitive Hydroxypropyl Chitin Sponge

To evaluate in vivo the local anesthetic efficacy of the drug-loaded implanted thermosensitive hydroxypropyl chitin sponges prepared in Example 1, guinea pig pin-prick assay was employed in this invention. Guinea pigs (around 300 g) were induced and maintained with isoflurane. Then the left backs of guinea pigs were shaved before treatment. One longitudinal skin incision with 1.5 cm in length was made by a scalpel at 2 cm left of ridge midline on the back of the guinea pig. Subsequently, one cuboid sponge about 0.5 g (1 cm×1 cm×0.5 cm) was put right in the middle of the incision below the skin. The incision was finally stitched up. 36 animals were randomly divided into six experimental groups with each of 6: group 1: BPV-loaded 0.8B %-HPCH (3.6 wt %) sponge, group 2: 0.8B %-collagen/HPCH (0.6/3.0 wt %) sponge, group 3: 1.2B %-HPCH (3.6 wt %) sponge, group 4: 2.4B %-HPCH (3.6 wt %) sponge, 0.8%-BPV group 5 (0.8 wt % BPV solution) and sham-operated group 6. As for sham-operated group, the incision was made and stitched up without placing a sponge. For BPV solution group, a volume of 0.5 mL 0.8% BPV solution was injected subcutaneously near the skin incision using 30-gage hypodermic needle. Eight pin-pricks distributed uniformly along the perimeter of a 1.5 cm diameter circle drawn around the placed sponge center were applied for the anesthetic effect evaluations using 24-gage needles at predetermined time interval (10 min, 30 min, 1 h, 2 h, 4 h, 6 h, 8 h, 10 h, 12 h, 14 h, 16 h, 18 h, 20 h, 22 h, 24 h, 28 h, 32 h, 36 h, 48 h). The number (n) of the points at which the guinea pigs did not respond to pin-prick indicated as local anesthetic effect was recorded. The nociceptive blockade score (N %) was calculated as following equation: N %=n/8×100%. Different drug loading content of the implanted thermosensitive hydroxypropyl chitin sponges was studied to test the subcutaneous local anesthetic blockade effect of postoperative pain. The complete inhibitions (no response, n=8, nociceptive blockade score 100% effect) of cutaneous trunci muscle reflex were observed at 10 min in all treatment groups containing drug bupivacaine. However, no inhibitions were observed anytime in the sham-operated group (nociceptive blockade score 0% effect).

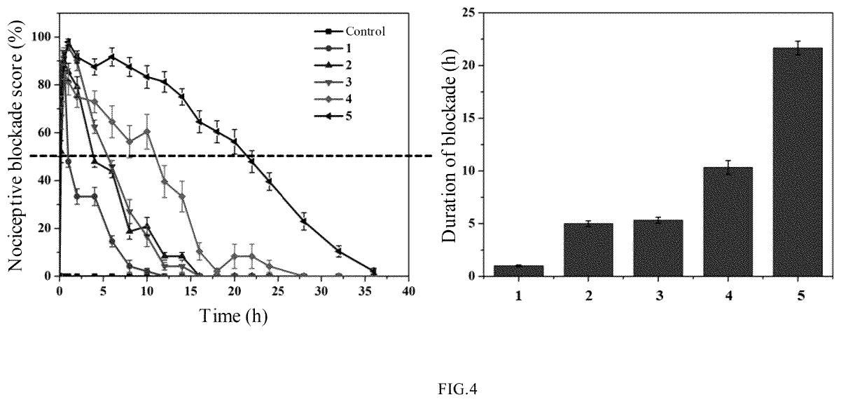

FIG. 4 is the in vivo nociceptive blockade score (a) and effective duration time of blockade (b) of 1 (0.8%-BPV solution) and sponges 2 (BPV-loaded 0.8%-HPCH (3.6 wt %)), 3 (BPV-loaded 0.8%-collagen/HPCH (0.6/3.0 wt %)), 4 (BPV-loaded 1.2%-HPCH (3.6 wt %)) and 5 (BPV-loaded 2.4%-HPCH (3.6 wt %)). Data are presented as mean±SD, n=6. **p<0.01, ***p<0.001. It can be seen from FIG. 4a that HPCH (3.6 wt %) sponge possessed the best inhibition effect under the same 0.8% BPV content, the same trend as in vitro drug sustained release data shown in Example 1. And the local anesthetic blockade time increased obviously with the increase of the drug loading content from 0.8% to 2.4%. For a clear comparison, the effective duration of blockade (defined as the time with nociceptive blockade score≥50%) of different sponges was demonstrated in FIG. 4b. As seen in this figure, the effective duration of blockade of BPV solution group 0.8%-BPV, 0.8% B-collagen/HPCH (0.6/3.0 wt %) and 0.8% B-HPCH (3.6 wt %) sponge group were 1 h, 5 h, 5.33 h, respectively, for the same 0.8% BPV drug content, the effective blockade time of 0.8% B-HPCH (3.6 wt %) sponge was 5.33 to that of BPV solution group. The effective blockade time for 1.2%-HPCH (3.6 wt %) sponge and 2.4%-HPCH (3.6 wt %) sponge was prolonged to 10.33 h and 21.67 h, respectively.

Histological evaluation was carried out to evaluate the biocompatibility of the BPV-loaded HPCH sponge implant through H&E staining. Some inflammatory cells including primarily macrophages and lymphocytes only existed in the surrounding tissues of sponges but did not penetrate into the skin and subcutaneous tissue layers for all groups implanted after 7 days. This indicated the inflammatory factors were limited to a certain range with mild inflammatory response. Also, our findings suggested that the inflammatory response of the BPV-loaded sponges implanted was not significantly enhanced with the increase of BPV content up to 2.4 wt BPV (2.4% B-HPCH (3.6 wt %)). This also implied that bupivacaine-loaded hydroxypropyl chitin sponges controlled the burst release of the drug effectively. Thus the bupivacaine hydrochloride (BPV) loaded thermosensitive hydroxypropyl chitin (HPCH) sponges prepared by a simple solvent-free green process in this invention could achieve postoperative long-term local anesthetic and analgesic effect without obvious tissue structural damage.

The concentration of the thermosensitive hydroxypropyl chitin ranging between 0.5-6 wt % in place of above 2 wt %, 3 wt % or 3.6 wt %, the concentration of local anesthesia within a range of 0.1-5%, and the addition concentration of hyaluronic acid or collagen being between 0.1% and 1%, the local anesthetic and analgesic drug being a mixture selected from any one or more of procaine, bupivacaine, levobupiyacaine, tetracaine, ropivacaine, etidocaine, articaine, lidocaine, mepivacaine, trimecaine, prilocaine or oxethazaine, and their salts and prodrugs, which can replace the ropivacaine or bupivacaine in the above experiment, resulted in the similar long-acting sustained-release local anesthesia effect. When the concentration of the thermosensitive hydroxypropyl chitin ranging between 1-4 wt %, and 0.2-0.8% hyaluronic acid auxiliary components contained, the local anesthetic and analgesic drug selected from 0.5-3% ropivacaine hydrochloride, the thermosensitive modified chitin sponge drug-loaded implanted sustained-release system has a better long-acting sustained-release local anesthesia effect.

The anti-inflammatory and antibacterial drugs include but are not limited to non-steroidal anti-inflammatory drugs, steroidal anti-inflammatory drugs or antibacterial drugs, selected from any one or more of aspirin, acetaminophen, indomethacin, naproxen, nabumetone, diclofenac, ibuprofen, nimesulide, rofecoxib, celecoxib, dexamethasone, prednison acetate, cortisone. In combination of any one or more of them with local anesthetic drug can replace the ropivacaine or bupivacaine in the above experiment, resulting in the similar long-acting sustained-release effect.

Different molar degrees of hydroxypropyl substitution (MS between 0.53-1.23) of thermosensitive HPCH samples with low degree of deacetylation (DA between 0.80-0.95) replace thermosensitive hydroxypropyl chitin above, resulting in the similar long-acting sustained-release effect. A combination of any one or more of thermosensitive hydroxypropyl chitin, thermosensitive carboxymethyl chitin, thermosensitive hydroxyethyl chitin, thermosensitive hydroxybutyl chitin, thermosensitive quaternized chitin or thermosensitive hydroxybutyl chitosan replace thermosensitive hydroxypropyl chitin above, resulting in the similar long-acting sustained-release effect.

This thermosensitive modified chitin sponge drug-loaded implanted sustained-release system is suitable to be applied for pain control of long-acting local anesthetic and analgesic drug or anti-inflammatory and antibacterial drug sustained-release at least 1 day after local surgical operations, including but not limited to, laparotomy, orthopaedic surgery, gynecologic surgery, thoracotomy, or abdominal surgery. It will be implanted subcutaneously before suturing after operation, such as open inguinal hernia repair, abdominal hernia repair, abdominal plastic surgery, for pain control of long-acting local anesthetic and analgesic or anti-inflammatory and antibacterial purpose.

At last, it should be noted that the above embodiments are only for illustrating the technical solution of the disclosure but not limiting the protective scope of the disclosure. Although the disclosure is described in detail with reference to preferred embodiments, those skilled in the art should understand that modifications or equivalent replacements can be made to the technical solution of the disclosure without departing from the essence and scope of the technical solution of the disclosure.

What is claimed is:

1. A thermosensitive modified chitin sponge drug-loaded implanted sustained-release system, wherein the system comprises the following components: a spongy matrix, the matrix comprising at least one thermosensitive modified chitin polymer, and at least one drug, the drug being a local anesthetic and analgesic drug and/or an anti-inflammatory and antibacterial drug, and the drug being uniformly dispersed in the matrix.

2. The thermosensitive modified chitin sponge drug-loaded implanted sustained-release system according to claim 1, wherein the gel transition temperature of the thermosensitive modified chitin is in the range of 1˜36° C., the thermosensitive modified chitin is a combination of any one or more of thermosensitive hydroxypropyl chitin, thermosensitive carboxymethyl chitin, thermosensitive hydroxyethyl chitin, thermosensitive hydroxybutyl chitin, thermosensitive quaternized chitin or thermosensitive hydroxybutyl chitosan, the concentration of the thermosensitive modified chitin in the matrix is 20-100% by mass.

3. The thermosensitive modified chitin sponge drug-loaded implanted sustained-release system according to claim 2, wherein the thermosensitive modified chitin is thermosensitive hydroxypropyl chitin with molar degrees of hydroxypropyl substitution between 0.53-1.23, degree of acetylation between 0.80-0.95 and the gel transition temperature of 10-30° C.

4. The thermosensitive modified chitin sponge drug-loaded implanted sustained-release system according to claim 1, wherein the spongy matrix also contains a combination of any one or more of collagen, hyaluronic acid, polyaspartic acid, polyglutamic acid, their amount in total in the spongy matrix is 0-80% by mass.

5. The thermosensitive modified chitin sponge drug-loaded implanted sustained-release system according to claim 1, wherein the local anesthetic and analgesic drug is an amino amide anaesthetic, an amino ester anesthetics and mixtures thereof, selected from any one or more of procaine, bupivacaine, levobupivacaine, tetracaine, ropivacaine, etidocaine, articaine, lidocaine, mepivacaine, prilocaine and oxethazaine, and their salts and prodrugs.

6. The thermosensitive modified chitin sponge drug-loaded implanted sustained-release system according to claim 5, wherein the concentration of local anesthesia is from 0.5 wt % to 3 wt %, the local anesthesia is selected from bupivacaine or ropivacaine.

7. The thermosensitive modified chitin sponge drug-loaded implanted sustained-release system according to claim 1, wherein the anti-inflammatory and antibacterial drug is a non-steroidal anti-inflammatory drug, steroidal anti-inflammatory drug or an antibacterial drug, selected from any one or more of aspirin, acetaminophen, indomethacin, naproxen, nabumetone, diclofenac, ibuprofen, nimesulide, rofecoxib, celecoxib, dexamethasone, prednison acetate, cortisone.

8. The thermosensitive modified chitin sponge drug-loaded implanted sustained-release system according to claim 1, wherein the spongy matrix also contains coating on the outer surface, the coating material selected from any one or more of dodecanol, tetradecanol, hexadecanol, octadecanol, dodecanoic acid, hexadecanoic acid, octadecanoic acid, the amount of coating materials in the spongy matrix is 2-20% by mass.

9. A preparation method of a thermosensitive modified chitin sponge drug-loaded implanted sustained-release system, comprising the following steps:
(1) dissolving thermosensitive modified chitin in normal saline or alkaline water below the gel transition temperature to obtain solution A;
(2) dissolving the drug or the drug hydrochloride into normal saline or acidic water to obtain solution B; and
(3) mixing the solution A with the solution B at a low temperature to prepare a mixed sponge precursor solution, and adjusting pH to 6.0-8.0 to obtain a homogenous mixed solution or even dispersion, being put in the cold storage chamber to defoam;
(4) the mixture from step (3) being added to a cubic mold for freezing and freeze drying, resulting in the thermosensitive modified chitin sponge drug-loaded implanted sustained-release system.

10. The method of claim 9, wherein the thermosensitive modified chitin is thermosensitive hydroxypropyl chitin, thermosensitive carboxymethyl chitin, thermosensitive hydroxyethyl chitin, thermosensitive hydroxybutyl chitin, thermosensitive quaternized chitin or thermosensitive hydroxybutyl chitosan or their combination and the concentration of the thermosensitive modified chitin used is from 0.5 wt % to about 6 wt %.

11. The method of claim 10, wherein the thermosensitive modified chitin is thermosensitive hydroxypropyl chitin and the concentration of the thermosensitive hydroxypropyl chitin used is from 1.0 wt % to about 4.0 wt %.

12. The method of claim 9, wherein the step (1) specifically comprises: dissolving thermosensitive modified chitin in alkaline water below the gel transition temperature to obtain solution A with pH around 8-14 and the concentration of the thermosensitive modified chitin from 0.5 wt % to 6 wt %; wherein the step (2) specifically comprises: dissolving the drug or the drug hydrochloride into acidic water to obtain solution B with the drug concentration from 0.1 wt % to 5 wt %, and acidic water is referred to one of the aqueous HCl solution, the aqueous H₂SO₄ solution or acetic acid solution.

13. A preparation method of a thermosensitive modified chitin sponge drug-loaded implanted sustained-release system according to claim 6, wherein the coating on the sponge outer surface is sprayed by a liquid layer, which can be dried or solidified to form a solid thin layer on the spongy matrix.

14. The method of claim 13, wherein the coating material used is a homogeneous liquid after heating or dissolving in volatile solvent, which can form a solid thin layer on the spongy matrix by cooling to solidify from liquid to solid state or by evaporation to remove solvent.

15. The method of claim 12, wherein the local anesthetic and analgesic drug is an amino amide anaesthetic, an amino ester anesthetics and mixtures thereof, selected from any one or more of procaine, bupivacaine, levobupivacaine, tetracaine, ropivacaine, etidocaine, articaine, lidocaine, mepivacaine, prilocaine and oxethazaine, and their salts and prodrugs.

16. The method of claim 9, wherein the step (3) specifically comprises: mixing the solution A with the solution B at a low temperature to prepare a mixed sponge precursor solution, and adjusting pH to 7.0-7.5 to obtain a homogenous mixed solution or even dispersion, being put in the cold storage chamber to defoam.

17. Use of the thermosensitive modified chitin sponge drug-loaded implanted sustained-release system according to claim 2 for a drug for long-acting local anesthesia, analgesia, anti-inflammation and itching relieving.

18. Use of the thermosensitive modified chitin sponge drug-loaded implanted sustained-release system according to claim 3 for a drug for long-acting local anesthesia, analgesia, anti-inflammation and itching relieving.

19. Use of the thermosensitive modified chitin sponge drug-loaded implanted sustained-release system according to claim 1 for a drug for block of peripheral nerves or control of postoperative wound pain.

20. Use of the thermosensitive modified chitin sponge drug-loaded implanted sustained-release system according to claim 5 for a drug for block of peripheral nerves or control of postoperative wound pain.
